(19) United States
(12) Patent Application Publication
(10) Pub. No.: US 2002/0156019 A1
(43) Pub. Date: Oct. 24, 2002

(54) Method of optimizing growth hormone replacement

(75) Inventor: Michael B. Foster, Phoenix, AZ (US)

Correspondence Address: Wood, Herron & Evans, L.L.P., 2700 Carew Tower, 441 Vine Street, Cincinnati, OH, 45202-2917, US

(73) Assignee: Renasci, Inc., d/b/a Renasci Anti-Aging Center

(21) Appl. No.: 09/838,968

(22) Filed: Apr. 20, 2001

(51) Int. Cl. A61K 38/18

(52) U.S. Cl. 514/21

(57) ABSTRACT

A method to replenish human growth hormone (hGH) in a human adult. A initial daily dose of hGH is administered for three to four weeks, then an individualized maintenance dose is determined by determining the individual's response to serially increased doses of the initial dose. The maintenance dose is then administered, usually in a microsphere formulation so that monthly dosing is possible. The method is useful to alleviate some of the effects of aging in mature adults.

### FIELD OF THE INVENTION

The invention relates to a method to determine therapeutic regimens of human growth hormone administration in adults, particularly as an anti-aging therapy.

### BACKGROUND

The aging process in humans has physiological and psychological manifestations. In the musculoskeletal system, bone density, muscle mass, and lean body mass decrease. Fat body mass increases. Serum lipid levels change, for example, the ratio of “good” and “bad” cholesterol changes. Skin tone and elasticity decrease. Cerebral function decreases. Sexual function decreases.

It is known that administration of human growth hormone (hGH) can reduce, at least to some degree, the above-mentioned effects of aging. Human GH is a polypeptide that is naturally produced by the pituitary gland. Human GH drives the process of normal rapid growth during childhood and adolescence, regulating a variety of functions in virtually all cell types involved in the process. The effects of hGH are most easily observed as growth in muscle, bone, and skin.

Human GH has been used clinically to treat children with growth deficiencies since the 1970s. In the past, the only way to obtain hGH was to isolate it from cadavers, leading to safety concerns because of possible contamination and disease transmission. Now, hGH is synthesized in ultrapure form using recombinant techniques, and can be safely administered.

Besides treating growth deficiencies in children, hGH received approval by the Food and Drug Administration (FDA) in 1997 for use in the treatment of growth hormone deficiency states in adulthood, either as an isolated hormone deficiency or as part of a global pituitary deficiency profile. The approved indication requires that the adult recipient of hGH have either manifested the deficiency in childhood or adolescence, or have a specific, identifiable cause of the deficiency in pituitary function such as head trauma, surgery, irradiation, etc.

It is known that the level of hGH production declines with age, so that the amount of hGH in a 40-50 year old male is less than one-half of the level in an 18-25 year old male. As knowledge of the benefits of hGH replacement therapy become widespread, the use of hGH in adults continues to increase. However, treatment with higher doses of hGH (doses that are two to three times the mean doses reported in most of the literature regarding GH deficient patients) may produce troublesome side effects. Examples of such side effects include edema, joint and muscle pain, and entrapment defects as occurs in carpal tunnel syndrome. These effects have been reported in about one-third of participants in the small number of clinical trials employing doses of hGH that are much larger than the typical doses reported in the treatment of GH deficient adult patients. Furthermore, the FDA approved indication for hGH treatment in adults excludes this recognized age-related decline in GH secretion as qualifying under the definition of GH deficiency.

The dose and treatment regimen of hGH in adults, however, still remains problematic. No scientifically validated standards for induction and maintenance phases of therapy have been promulgated. The single method that has so far been established uses only a subtherapeutic dose of hGH. While this dose assures that recipients will avoid troublesome side effects, it does not attend to the individual needs and responses of recipients. Moreover, this method uses hGH in a regimen that has no hGH enhancing effects; rather, it employs several low potency anabolic steroid hormones in pharmacological doses, which collectively have the effect of only mimicking some responses to hGH.

Thus, methods that currently exists for treating adults with hGH use either an ineffective dose of hGH, which is masked by the side effects of high doses of anabolic steroids, or produce an unacceptably high risk of troublesome side effects. While it has been suggested that side effects may be minimized by carefully monitoring the dose of GH and adjusting it to produce optimum levels of insulin like growth factor 1 (IGF-1) (Carter Clinics in Geriatric Medicine, Vol. 11, pp. 735-748, November 1995), such a method has not been reported. In addition, such methods lack sufficient attention to individual needs and responses of the recipients. Thus, a method to optimize hGH replacement therapy in adults is needed.

### SUMMARY OF THE INVENTION

The inventive method provides for a composition that is administered to an adult in order to provide anti-aging effects. The method involves administering a composition of human growth hormone (hGH) that lacks other hormones or other bioactive compounds. The method employs an inductive dose of hGH, and considers the individual's own response to daily doses of hGH to determine the desired maintenance dose unique for that individual.

No other known methods provide for individualized dosing of a composition that contains only hGH as the active component. Furthermore, in contrast to methods which require daily or even more frequent dosing, the inventive method permits the maintenance dose to be administered as infrequently as on a monthly basis by dosing hGH in a time-released formulation, such as a microsphere. This provides convenience to the individual and removes the unpleasantness of frequent injections.

The invention will be further appreciated in light of the following detailed description and examples.

### DETAILED DESCRIPTION

A method is disclosed to replenish the age-related decline in human growth hormone (hGH) in adults by administering an individualized dosing regime of hGH in the absence of any other bioactive compounds. In the method, the individual initially receives incrementally increasing doses of hGH (inductive dose), while undergoing physiological and sociological assessment to determine the effect of hGH. Bases on these outcomes, a maintenance dose to achieve the desired hGH replenishment for the individual is then determined. Thereafter, the individual receives this maintenance dose of hGH, either on a daily or monthly basis, depending upon his or her preference. Outcomes of this method of individualized hGH therapy include increased bone density, muscle and lean body mass, decreased fat body mass, improvement in serum lipid levels, for example, the ratio of “good” and “bad” cholesterol, improvement in skin tone and elasticity, improved cerebral function, improved sexual function, and an improved general sense of well being.

The individual undergoing therapy is an adult, either male or female and typically at least 40 years old, who is in a general state of good health. Good health is assessed by a preliminary examination, including a complete medical history with a complete list of all medications taken regularly. A physical examination is also performed, including hematological and chemical panels to determine overall health, biological age and, in particular, current levels of various hormones such as hGH, testosterone, estrogen in women, and insulin like growth factor 1 (IGF-1). Hormone levels are charted and compared to normal baseline levels for biological age to determine the extent of their depletion, and as a baseline from which to assess therapeutic outcomes. Adults treated with thyroid hormone, testosterone, and estrogen are amenable to treatment according to the inventive method, and the parameters in their preliminary evaluation will be valid as long as the dose of the medication is stable. If clinical assessment indicates levels of testosterone or estrogen that are too high, or levels of thyroid hormone that are either too low or too high, the dose must be adjusted and blood levels of the offending medication must be permitted to reach a new equilibrium before proceeding with the inventive method.

An individual-specific treatment with hGH is then initiated. A stable liquid hGH formulation is used to gradually replenish hGH. This allows an optimum physiological replacement dose of hGH to be administered to the individual with a risk of side effects to be within an acceptable range. In contrast to known methods, the inventive method does not administer adjunctive agents such as other hormones or bioactive compounds such as melatonin or vitamin B12.

The hGH hormone administered is a commercially available synthetic peptide (Nutropin®, Genentech, San Francisco, Calif.), produced by recombinant molecular biology techniques. Administration of hGH is by parenteral means. In one embodiment, hGH is administered by subcutaneous injection. Injection may be in the arm, leg, stomach, buttock, or hip.

A daily injection of hGH is administered in an initial phase of therapy. A stable solution of hGH (Nutropin AQ®, Genentech, San Francisco, Calif.) is administered at a dose of about 2 μg/kg/day in males and about 4 μg/kg/day in females. Daily injections of this initial dose are continued for about three to four weeks.

The initial dose of hGH is then titrated to an adjusted, or maintenance, dose. The maintenance dose is that which produces the desired level of hGH replenishment for that individual. Maintenance doses are typically about 10-14 μg/kg/day for males, and 14-20 μg/kg/day for females, and are determined by physical response and attainment of desired levels of IGF-1. Since IGF-1 is produced in response to growth hormone, the level of IGF-1 serves as a mediator of the anabolic effects of hGH therapy in adults, as well as statural response in children. Measurement of circulating levels of IGF-1 provides an accurate index of an integrated measure of GH level and effect. Furthermore, whereas GH levels are very volatile and difficult to interpret clinically, IGF-1 levels are exceedingly stable and can be assayed in blood samples that are drawn at any time of day.

The maintenance dose of hGH is determined by evaluating the individual's response to serially increased doses of hGH, usually over one to two months. The dose is adjusted at about two to four week intervals, and in a range equal to that of the initial dose. For example, a male receiving an initial dose of 2 μg/kg/day would receive a serially increased dose of 4 μg/kg/day for two to four weeks, then a dose of 6 μg/kg/day for two to four weeks, then a dose of 8 μg/kg/day for two to four weeks, etc., until the maintenance dose is achieved. A female receiving an initial dose of 4 μg/kg/day would receive a serially increased dose of 8 μg/kg/day for two to four weeks, then a dose of 12 μg/kg/day for two to four weeks, etc., until the maintenance dose is achieved.

Once the maintenance dose is achieved, a monthly dose of hGH is administered. This may be done by administering hGH in a time-released formulation, such as a microsphere formulation (Nutropin Depot®, Genentech, San Francisco, Calif.). To calculate the monthly dose, individualized bioavailability data are determined, since the microsphere formulation has 10-20% less bioavailability than daily dose formulations. While monthly administration regimens are available, that is, only one injection per month, and in fact may be preferred because of scheduling or other demands, a daily regimen is equally effective for those recipients desiring daily administration for psychological or other factors.

Outcome assessment of therapy is based on one or more combinations of several objective and/or subjective parameters. Objective outcome parameters include physical assessment such as total body weight, standing height, body composition as measured by electrical impedance or another type of measurement, tolerance to exercise, biochemical assessment such as measurement of blood levels of IGF-1 and other growth hormone-dependent parameters such as insulin like growth factor binding proteins 1-4 (IGF-1, IGF-2, IGF-3, and IGF-4, particularly IGF-3 and its acid labile subunit (ALS)), and psychological assessment such as memory tests. The measurement of each of these parameters is known to one skilled in the art. Subjective outcome parameters include responses to questionnaires concerning, for example, improvement in sexual function and a general sense of well being.

The following examples illustrate the inventive method.

### EXAMPLE 1

The subject was a 78 year old male retired professional. Throughout his career he had been a leader in his community and had been active in a variety of pursuits, ranging from daily exercise to an avid practice of several creative arts. As he progressed into his sixties, he found that he was experiencing an unacceptable decline in his physical capabilities. His capacity for exercise began to diminish and, because of his meticulous attention to his fitness, he was aware of progressive deterioration of his lean body mass. He also began to experience several orthopedic problems, including a change in his posture, which were very distressing to him. He was medically sophisticated, and so he turned to the remedies which were available to him, but he experienced frustratingly meager responses. Finally, he began to experience a decline in his creativity, which increased his sense of urgency in seeking an effective response to the ravages of aging.

After lengthy discussion of the possible alternatives, he elected treatment with an agent employing both testosterone and dehydroepiandrosterone (DHEA), purported to stimulate native growth hormone secretion. The agent was completely ineffective in producing the desired response of a renewal of his physical capacities. The agent also did not produce a biochemical response in the form of restoration of his index of growth hormone activity to a level commensurate with robust good health for an adult. At this point in his history, and without achieving a satisfactory response, the subject began administration of human growth hormone using the inventive method.

A baseline IGF-1 level of 119 ng/ml confirmed that his level of GH secretion was low. Daily subcutaneous hGH therapy was begun at a dose of 150 μg/day (about 2.5 μg/kg/day). During the induction of hGH therapy, his self-prescribed daily oral supplement of DHEA was eliminated from his routine.

After three weeks of therapy, in the absence of side effects and with his IGF-1 level elevated to 184 ng/ml, his hGH dose was increased to 300 μg/day (about 5 μg/kg/day). After an additional three weeks of therapy, his IGF-1 level rose to 237 ng/ml. Still in the absence of side effects, his daily hGH dose was increased to 450 μg/day (about 7.5 μg/kg/day). After a third three week interval, there were still no adverse effects and his IGF-1 level rose to 270 ng/ml. His daily hGH dose was increased to 600 μg/day (about 10 μg/kg/day). After a fourth three week interval, there were still no adverse effects and his IGF-1 level rose to 305 ng/ml. His IGF-1 level was stable above 300 ng/ml on a daily dose of 600 μg/day.

Once the appropriate therapeutic dose of human growth hormone was identified and administered, continued hGH therapy produced astounding physical changes. The subject's exercise capacity was the first parameter to undergo a noticeable change. Both endurance in aerobic exercise and strength in resistance exercise showed significant improvement. Posture was the next parameter to show a credible response. The change was noticeable even to casual observation by friends and associates who encountered the subject in his day-to-day life. Finally, restoration of lean body mass was exhibited in facial appearance and in measurement of chest, abdomen and upper arm circumference. The unsolicited response from observers who were unaware of the therapy was that he appeared more robust, fitter and, in a word, younger.

An unanticipated result of the therapy was that the subject began to experience an undoubted increase in his creative productivity. This did not appear to be solely the result of an increase in his physical capacity for work. What the subject described as his interior life was more vibrant. Ideas came in torrents as they had during his younger years, and he felt the restoration of his zeal to create. Simultaneously, he experienced a reawakening of his sense of sexual competence. The subject has regained the scope of interests and activities that characterized his middle years. Having known him over a period of a decade and a half, the word that comes to mind is “rejuvenation”.

### EXAMPLE 2

The subject was a male in his late 50's who had experienced significant concomitant fractures in both the tibia and fibula. In spite of intensive conventional therapy, the injury had failed to heal completely. An evaluation showed a baseline IGF-1 level of 74 ng/ml, revealing clear evidence of his lack of the beneficial influence of growth hormone. Therapy with hGH was begun in an attempt to enhance his healing.

Since his IGF-1 level was quite low, therapy with hGH was begun at a very low dose. The initial hGH dose was 100 μg/day, calculated on the basis of about 1.2 μg/kg/day). This produced an IGF-1 level of 91 ng/ml after a three week interval. The second hGH dose was 200 μg/day. After an additional three weeks of therapy, his IGF-1 level rose to 126 ng/ml. Still in the absence of side effects, his daily hGH dose was increased to 400 μg/day, with subsequent doses adjusted at three week intervals in increments equal to his second dose (that is, in increments of 200 μg/kg) because he remained free of any side effects through the first two three-week intervals. After a hGH dose interval of 400 μg/day, his IGF-1 level rose to 235 ng/ml. After a hGH dose interval of 600 μg/day, his IGF-1 level rose to 294 ng/ml. After a dose interval of 800 μg/day, his IGF-1 level rose to 321 ng/ml. He experienced no adverse side effects and required no ancillary hormone replacement therapy.

Within a few weeks of achieving an IGF-1 level above 300 ng/ml, a measurable improvement in the healing of his fractures was apparent. The therapy was continued until the injury had healed completely. During the period of initial therapy, the subject reported the restoration of a joie de vivre that had been lacking for a period of years. His physical therapy became enjoyable and, with the healing of his injury, he embarked upon a much more active lifestyle.

With the complete resolution of his original injury, the subject has elected to continue his therapy with hGH because of the improvement in his quality of life. He has abandoned his sedentary habits and is exploring a variety of new physical activities. He looks and feels more youthful, and the people in his life who are unaware of his therapy are surprised at the change that has taken place in his approach to living. He has brought innovations into his already successful business and he is finding new outlets for his creative energies. With complete healing of his initial injury, the option of discontinuing therapy has come under discussion, but he stated that he has no intention of relinquishing his hold on an enhanced life.

### EXAMPLE 3

The subject was a woman in her early 50's who had experienced problems with infertility, but who considered herself otherwise healthy. She had been plagued by a progressive decline in her endurance and in her enthusiasm for the normal range of activities in her life. She inquired about the possibility that a low level of growth hormone secretion might be contributing to the problems that she had been experiencing. She proved to have an IGF-1 level of 176 ng/ml, indicating a low level of growth hormone secretion. In light of that finding, she was ready to consider herself a candidate for hGH therapy.

Therapy with hGH was initiated at a dose of 200 μg/day (about 4 μg/kg/day). The hGH dose was raised in increments of 200 μg/day at four-week intervals to a maintenance hGH dose of 800 μg/day (about 15 μg/kg/day). These hGH doses produced IGF-1 levels as follows: an IGF-1 level of 194 ng/ml with a hGH dose of 200 μg/day, an IGF-1 level of 208 ng/ml with a hGH dose of 400 μg/day, an IGF-1 level of 243 ng/ml with a hGH dose of 600 μg/day, and an IGF-1 level of 297 ng/ml with a hGH dose of 800 μg/day. Her IGF-1 level was stable in the range of 300 ng/ml on a daily dose of 800 μg/day.

With her IGF-1 levels in the normal range, she began to experience a return to her former level of physical activity and a resurgence in enthusiasm for the other interests that had comprised her active and productive life. The subject had already experienced the menopause, and she was taking appropriate hormone replacement therapy. The introduction of hGH therapy did not require altering her other hormone replacement therapy in any way, but did serve to enhance her sense of well being which had been only partially restored by the traditional therapy.

The subject is now participating in daily aerobic exercise, including serving as an instructor in several classes per week. She has experienced an awakening of new enthusiasm for her career, and is making plans to expand the scope of her professional activities. She is monitoring her body composition and, though her weight has actually remained unchanged, she is leaner and stronger than she has been at any point in her life after adolescence.

It should be understood that the embodiments of the present invention shown and described in the specification are only preferred embodiments of the inventor who is skilled in the art and thus are not limiting in any way. Therefore various changes, modifications or alterations to these embodiments may be made or resorted to without departing from the spirit of the invention and the scope of the following claims.

What is claimed is:

1. A method of replenishing human growth hormone (hGH) in an adult human comprising administering an agent consisting essentially of recombinant hGH in an individualized dose to replenish hGH, said individualized dose determined by
(1) determining a response of said human to an initial dose of said agent administered on a daily basis,
(2) thereafter determining a response of said human to serially increased doses of said agent administered on a daily basis,
(3) selecting said dose of agent from (2) producing an optimal replenishment to administer as a maintenance dose, and
(4) thereafter administering said dose from (3) to replenish hGH.

2. The method of claim 1 wherein said maintenance dose is administered monthly.

3. The method of claim 2 wherein said dose comprises a microsphere formulation of said agent.

4. The method of claim 1 wherein said dose is administered daily.

5. The method of claim 1 wherein said human is a male and said maintenance dose is in the range of about 10-14 μg/kg/day.

6. The method of claim 1 wherein said human is a female and said maintenance dose is in the range of about 14-20 μg/kg/day.

7. The method of claim 1 wherein said response comprises increased insulin like growth factor levels.

8. The method of claim 1 wherein said human is a male and said initial dose is about 2 μg/kg/day.

9. The method of claim 1 wherein said human is a female and said initial dose is about 4 μg/kg/day.

10. A method of providing an adult human with human growth hormone (hGH) comprising
administering an agent consisting essentially of recombinant hGH to said human on a daily basis at an initial dose to produce an initial response to said agent,
thereafter administering at least one serially increased initial dose of said agent on a daily basis and evaluating said human's response to said serially increased dose to produce an individualized optimal response to said agent, and
thereafter administering said dose producing said optimal response as a maintenance dose.

11. The method of claim 10 wherein said optimal dose is administered monthly.

12. The method of claim 10 wherein bioavailability data are used to calculate said maintenance dose.

13. The method of claim 10 wherein said response is evaluated by evaluating a level of insulin like growth factor.

14. A method of optimizing human growth hormone (hGH) replacement in an adult human comprising
(1) administering an initial dose of hGH in the range of about 2 μg/kg/day hGH to about 4 μg/kg/day on a daily basis for about three to four weeks and determining insulin like growth factor 1 (IGF-1) levels,
(2) thereafter administering serially increasing doses of said initial hGH dose on a daily basis for about three to four weeks and determining IGF-1 levels,
(3) selecting said hGH dose from (2) producing optimal hGH replenishment to administer as a maintenance dose, and
(4) thereafter administering said maintenance dose in the range of about 10 μg/kg/day hGH to about 20 μg/kg/day hGH to said individual.

15. The method of claim 14 wherein said maintenance dose is administered monthly.

16. The method of claim 15 wherein said maintenance dose comprises hGH formulated in microspheres.

17. The method of claim 16 wherein bioavailability for said individual is determined before administering said maintenance dose.

18. The method of claim 14 wherein said maintenance dose is administered daily.
